(19) United States
(12) Patent Application Publication
(10) Pub. No.: US 2012/0029578 A1
(43) Pub. Date: Feb. 2, 2012

(54) Bio-Resorbable Capsule Containing Fenestrated Screw System For Osteoporotic Subject

(76) Inventor: Sean Suh, Plymouth Meeting, PA (US)

(21) Appl. No.: 13/018,636

(22) Filed: Feb. 1, 2011

Related U.S. Application Data
(60) Provisional application No. 61/301,930, filed on Feb. 5, 2010.

(51) Int. Cl. A61B 17/86; A61B 17/58

(52) U.S. Cl. 606/309; 606/93

(57) ABSTRACT

The invention encompasses compositions, methods, and kits, for fixing bone and/or repairing connective tissue associated with bone using a fenestrated bone screw. In certain embodiments, the invention encompasses a pedicle screw system including a bio-resorbable or implantable capsule containing cement and methods of use thereof for treating, preventing, and managing disorders in osteoporotic patients. In certain embodiments, the invention encompasses a fenestrated pedicle screw with cement containment skin that is useful in treating subjects in need of spine surgery due to neurologic deficit, deformity, or trauma, which subjects do not have sufficient time for the medical treatment of osteoporosis.

### FIELD OF THE INVENTION

The invention encompasses compositions, methods, and kits, for fixing bone and/or repairing connective tissue associated with bone using a fenestrated bone screw. In certain embodiments, the invention encompasses a pedicle screw system including a bio-resorbable or implantable capsule and methods of use thereof for treating, preventing, and managing disorders in osteoporotic patients. In certain embodiments, the invention encompasses a fenestrated pedicle screw including a bio-resorbable or implantable containment skin that is useful in treating subjects in need of spine surgery due to neurologic deficit, deformity, or trauma, which subjects do not have sufficient time for the medical treatment of osteoporosis.

### BACKGROUND OF THE INVENTION

The development of various internal spinal fixation devices to provide stability, reduce deformity, and enhance fusion rates has greatly influenced spinal surgery. The use of pedicle screw instrumentation has become increasingly popular and effective in the management of vertebral fractures, scoliotic deformities, metastatic disease, and degenerative disorders. The pedicle screw construct has three main advantages over other internal spinal fixation constructs: the ability to provide 3-column fixation, facilitate the instrumentation of short segments, and maintain anatomic or desired sagittal alignment. The key to the fixation, however, lies in the strength of attachment obtained by the screws in the pedicle and in the trabecular bone of the vertebral body.

Pedicle screws are the workhorse of spinal instrumentation for the adult spine, and screw attachment is a major concern in the osteoporotic patient. The effectiveness of pedicle screw-fixated instrumentation is critically dependent on the bone-screw interface. Pedicle screws rely primarily on cancellous bone for purchase, with the pedicle providing approximately 60% of the pullout strength.

The key determinant of pedicle screw performance is the strength of attachment to the spine, which is related to the quality of bone at the insertion site, with the strongest fixation achieved when the pedicle screw is placed in dense, good quality trabecular bone. Because age-related bone loss diminishes the integrity of the trabecular bone, pedicle screws that are inserted into osteoporotic patients will be at an increased risk for stripping during insertion and for loosening at a later date. Loosening at the bone-screw interface may render the pedicle inadequate for subsequent revision pedicle screw insertion, and the patient may be predisposed to recurrent instrumentation failure. Thus, a need exists for a method of augmenting and strengthening the bone-screw interface that will reduce the risk of pedicle screw loosening and increase the endurance limit for both virgin and revision pedicle screw fixation.

Failure of pedicle screws is of significant clinical importance, particularly to patients with osteoporosis or poor trabecular bone quality. The inventors have developed a bio-resorbable or implantable capsule containing cement pedicle screw system for treating, preventing and managing disorders in osteoporotic patients.

### SUMMARY OF THE INVENTION

The inventors have surprisingly found that the use of a containment skin or capsule in combination with a fenestrated screw overcomes the limitations associated in treating osteoporotic patients that require screw fixation, for example, pedicle screw fixation. The invention is particularly useful with dynamic spine systems anchored to the bone, which are subject to loosening or “windshield wiping” effects as the bone density of the treated subject decreases.

Accordingly, in one embodiment, the invention encompasses a fenestrated screw comprising one or more bio-resorbable or implantable containment capsules or skins that improve the fenestrated screw-bone attachment. The fenestrated screws of the invention are especially useful in subjects suffering from decreased bone density, for example, an osteoporotic patient.

In another embodiment, the invention encompasses methods for treating an subject requiring bone containment comprising:

a. creating a cavity in a bone, for example, vertebral bone;

b. inserting one or more bio-resorbable or implantable, containment skins into the cavity;

c. inserting bone cement into the cavity; and

d. inserting one or more screw(s) into the cavity.

In another embodiment, the invention encompasses methods for locking relative positions of a first and second vertebrae in a subject suffering from osteoporosis comprising:

a. providing a first fenestrated screw comprising a bio-resorbable or implantable capsule attached to the first vertebra;

b. providing a second fenestrated screw comprising a bio-resorbable or implantable capsule attached to the second vertebra;

c. engaging with the first fenestrated screw such that the second fenestrated screw is translatable toward and away from the first member to permit stabilized motion of the second vertebra with respect to the first vertebra, and

d. a first substantially elongate resilient member positioned within a cavity defined by engagement of the first and second fenestrated screw along a longitudinal axis of the cavity, such that the first and second fenestrated screws cooperate to keep the first resilient fenestrated screw substantially straight; and inserting a first locking component through a plurality of apertures formed in the first fenestrated screw and into contact with a portion of the second fenestrated screw disposed within the first member to lock the first stabilizer to substantially prevent translation of the second fenestrated screw toward and away from the first member, wherein the inserting step includes placing prongs of a clip in the apertures.

In another embodiment, the invention encompasses methods for treating an subject requiring bone containment comprising:

a. creating a cavity in a vertebral body;

b. inserting into the cavity a fenestrated screw including a bio-resorbable or implantable containment capsule; and

c. filling the bio-resorbable or implantable containment capsule through the apertures in the fenestrated screw with cement, for example, polymethylmethacrylate, to interdigitate with the cancellous bone, which allows the flexible containment capsule to conform to the cavity.

In certain embodiments, the fenestrated screw can be removed from the bone by an ultrasonic inducer probe, a mechanical removal device, or combinations thereof.

### BRIEF DESCRIPTION OF THE FIGURES

A more complete understanding of the present invention may be obtained by reference to the accompanying drawings, when considered in conjunction with the subsequent detailed description. The embodiments illustrated in the drawings are intended only to exemplify the invention and should not be construed as limiting the invention to the illustrated embodiments, in which:

FIG. 1 illustrates a non-limiting, exemplary fenestrated screw of the invention including a bio-resorbable or implantable containment capsule, wherein the fenestrated screw has a needle tip.

FIG. 2 illustrates a non-limiting, exemplary method of the application of the fenestrated screw of the invention. A cavity is created in a vertebral body. The fenestrated screw including a bio-resorbable or implantable containment capsule is inserted into the cavity. The bio-resorbable or implantable containment capsule is then filed with cement, for example, polymethylmethacrylate, which allows the flexible containment capsule to conform to the cavity.

FIG. 3 illustrates a non-limiting, exemplary fenestrated screw of the invention, wherein the fenestrated screw is fully threaded.

FIG. 4 illustrates a non-limiting, exemplary fenestrated screw of the invention including a bio-resorbable containment capsule that includes bone cement, wherein the fenestrated screw has a needle tip.

FIG. 5 illustrates a non-limiting, exemplary vertebral body cavity.

FIG. 6 illustrates a non-limiting, exemplary stepwise process for the creation of a vertebral body cavity, insertion of a bio-resorbable capsule, bone cement and pedicle screw.

FIGS. 7a and 7b illustrate a non-limiting, exemplary example of a fenestrated screw including a bio-resorbable capsule inserted in a vertebral cavity.

FIG. 8 illustrates a non-limiting, exemplary bio-resorbable collagen capsule or injectable coating gel.

FIG. 9 illustrates a non-limiting, exemplary cemented screw removal system.

FIG. 10 illustrates a non-limiting, exemplary bio-resorbable capsule contained cement pedicle system of the invention for osteoporotic patients.

FIG. 11 illustrates a non-limiting, exemplary embodiment of removal of a pedicle screw of the invention.

### DETAILED DESCRIPTION OF THE INVENTION

The invention generally encompasses a fenestrated screw comprising a bio-resorbable or implantable containment skin, gel, or capsule.

In certain exemplary embodiments, the fenestrated screw includes one or more apertures located on a first distal end of the fenestrated screw.

In certain exemplary embodiments, the fenestrated screw includes a second distal end optionally including a receiving segment. In other exemplary embodiments, the fenestrated screw comprises a receiving segment on a second distal end to allow attachment, for example, of a dynamic spine system, to the fenestrated screw. In certain exemplary embodiments, the receiving segment on the second distal end allows the fenestrated screw to be connected to a stabilizing system or device; or a removal device.

In certain exemplary embodiments, the containment skin encapsulates one or more apertures on the fenestrated screw.

In certain exemplary embodiments, the fenestrated screw is a pedicle screw.

In certain exemplary embodiments, the fenestrated screw is partially threaded.

In certain exemplary embodiments, the fenestrated screw is completely threaded.

In various embodiments, the containment skin is bio-resorbable, implantable or a combination of both. In certain exemplary embodiments, the bio-resorbable or implantable containment skin is comprised of, for example, collagen, poly-L-lactide (PLLA), poly-L/D-lactide (PLDLA; a mixture of poly-L-lactic acid with, for example, 30% of poly-D-lactic acid), polyanhydride, polyactide, polyglycolide, polyorthoester, polyphosphazene, calcium phosphate, hydroxyapatite, bioactive glass, tyrosine-derived polycarbonate, silicon, thermoplastic polycarbonate urethane (i.e., Bionate®), and mixtures thereof.

In certain exemplary embodiments, the containment skin is comprised of a bio-resorbable or implantable collagen capsule or injectable coating gel material.

In certain exemplary embodiments, the containment skin is comprised of a pressure-resistant material.

In certain exemplary embodiments, the containment skin is comprised of a flexible material to allow the containment skin to interdigitate with the cancellous bone.

In certain exemplary embodiments, the containment skin is comprised of a polymer material.

In certain exemplary embodiments, the stabilizing system or device comprises one or more of screws and cords; screws, cords, and spacers; screws and flexible rods; screws and rods with movable parts; flexible screws and flexible rods; screws and inflatable rods; and combinations thereof.

In certain exemplary embodiments, the removal device is an ultrasonic inducer probe. In other embodiments, the removal device is a mechanical removal device, for example, a bone trocar, which allows removal of an implant and cement bolus by grinding the cement away and removing the implant in a cylindrical like core piece.

In certain exemplary embodiments, the containment skin is coated with a bone growth agent. In certain exemplary embodiments, the bone growth agent comprises wherein the bone growth agent comprises insulin-like growth factor 1, basic fibroblast growth factor, transforming growth factor-β-1 (TGF-β), platelet-derived growth factor, bone-derived growth factors, arginine, bone morphogenetic protein (BMPs), LIM mineralization protein (LMP-1), Osf-1, and combinations thereof.

In certain exemplary embodiments, the containment skin is filled with cement which conforms to the vertebral cavity and allows attachment to a fenestrated screw to the vertebrae. In certain exemplary embodiments, the cement is polymethylmethacrylate.

In another embodiment, the invention encompasses methods of treating an subject requiring bone containment comprising:

a. creating a cavity in a bone;

b. inserting one or more containment capsules, preferably bio-resorbable or implantable capsule(s);

c. inserting bone cement into the cavity; and

d. inserting one or more screw(s) into the cavity.

In another embodiment, the invention encompasses methods of treating an subject requiring bone containment comprising:

a. creating a cavity in a bone;

b. inserting a fenestrated screw including a bio-resorbable or implantable containment capsule;

c. inserting bone cement through the fenestrated screw and into the bio-resorbable or implantable containment capsule, which is in the bone cavity.

In certain exemplary embodiments, the subject is a mammal.

In certain exemplary embodiments, the subject has osteoporosis.

In certain exemplary embodiments, the subject is a human.

In certain exemplary embodiments, the bone is cancellous bone.

In certain exemplary embodiments, the bio-resorbable or implantable capsule when filled with cement conforms to the cavity wall.

In certain exemplary embodiments, inserting one or more bio-resorbable or implantable capsule(s) and inserting bone cement into the cavity are achieved using a fenestrated screw comprising one or more aperture(s), wherein the bio-resorbable or implantable capsule(s) encapsulates one or more apertures on the fenestrated screw.

In certain exemplary embodiments, the fenestrated screw is a pedicle screw.

In certain exemplary embodiments, the fenestrated screw is partially threaded.

In certain exemplary embodiments, the fenestrated screw is completely threaded.

In certain exemplary embodiments, the method further comprises attaching to the fenestrated screw a stabilizing system or device comprising one or more of screws and cords; screws, cords, and spacers; screws and flexible rods; screws and rods with movable parts; flexible screws and flexible rods; screws and inflatable rods; and combinations thereof.

In another exemplary embodiment, the invention encompasses method for locking relative positions of a first and second vertebrae in a subject suffering from osteoporosis comprising:

a. providing a first fenestrated screw comprising one or more containment capsules, preferably bio-resorbable or implantable capsule(s) attached to the first vertebra;

b. providing a second fenestrated screw comprising one or more containment capsules, preferably bio-resorbable or implantable capsule(s) attached to the second vertebra;

c. engaging with the first fenestrated screw such that the second fenestrated screw is translatable toward and away from the first member to permit stabilized motion of the second vertebra with respect to the first vertebra, and

d. a first substantially elongate resilient member positioned within a cavity defined by engagement of the first and second fenestrated screw along a longitudinal axis of the cavity, such that the first and second fenestrated screws cooperate to keep the first resilient fenestrated screw substantially straight; and inserting a first locking component through a plurality of apertures formed in the first fenestrated screw and into contact with a portion of the second fenestrated screw disposed within the first member to lock the first stabilizer to substantially prevent translation of the second fenestrated screw toward and away from the first member, wherein the inserting step includes placing prongs of a clip in the apertures.

In another embodiment, the invention encompasses methods for treating an subject requiring bone containment comprising:

a. creating a cavity in a vertebral body;

b. inserting into the cavity a fenestrated screw including a bio-resorbable or implantable containment capsule; and

c. filling the bio-resorbable or implantable containment capsule through the apertures in the fenestrated screw with cement, for example, polymethylmethacrylate, to interdigitate with the cancellous bone, which allows the flexible containment capsule to conform to the cavity.

### Fenestrated Screws of the Invention

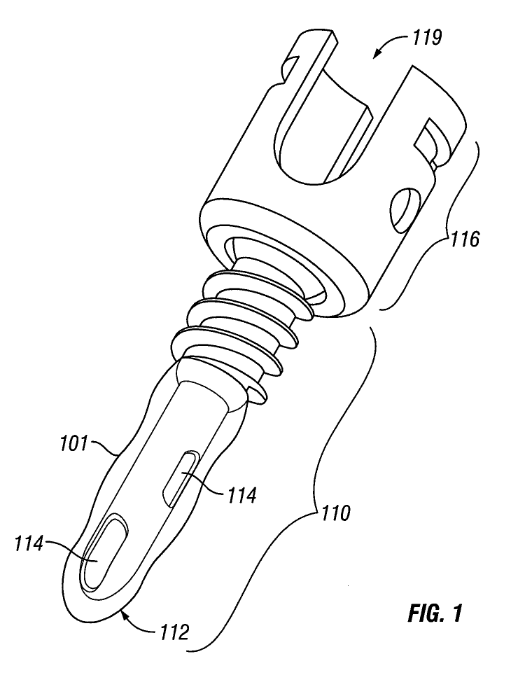

The invention generally encompasses a bone screw as illustrated in FIG. 1 comprising: (a) a shaft 110 optionally comprising at a first distal end including an at least partially threaded or needle-tip fenestrated body for fixation to a bone and optionally a receiving segment 116 extending from a second distal end, for example, to receive a rod implant.

In certain embodiments, the fenestrated screws of the invention include a bio-resorbable or implantable containment capsule or skin 101 covering either partially or completely the threaded or needle-tip region of the fenestrated screw. As used herein and unless otherwise indicated “bio-resorbable” means that the containment capsule or skin breaks down over a finite period of time due to the chemical/biological action of the body. Preferably, complete resorption occurs within about 5 years. More preferably within about 3 years. This breakdown is at a rate allowing the repair device to maintain sufficient integrity while the soft tissue or bone heals: surgical repair devices formed of materials which are resorbed too quickly may fail when compressive, tensile or flexural loads are placed on them before the tissue or bone has fully healed. In certain embodiments, advantages of using bio-resorbable materials over non-bio-resorbable materials (e.g., metals) are that they encourage tissue repair and further surgery is not required to remove them. In addition, there is the issue of stress-shielding: tissues like bone tend to grow well in regions where there is a prevalence of high stress. If the stress is reduced or removed, because, for example, an implant is bearing the load, then the tissue may tend to recede around it resulting in loosening over the longer term. Implanted bio-resorbable materials do not tend to give rise to adverse effects due to stress-shielding. In certain embodiments, interdigitation of an implantable containment capsule with cancellous bone can mitigate the adverse effects of stress-shielding. As used herein, the term “implantable” means that the containment capsule or skin remains intact in the vertebral cavity without causing any adverse or toxic effects to the subject.

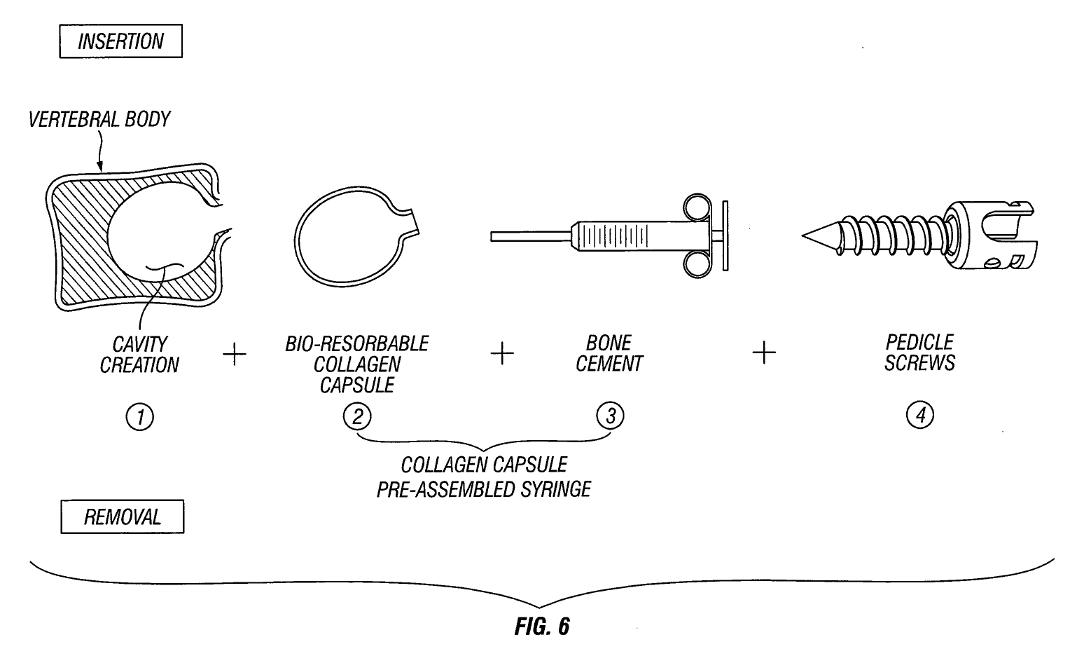

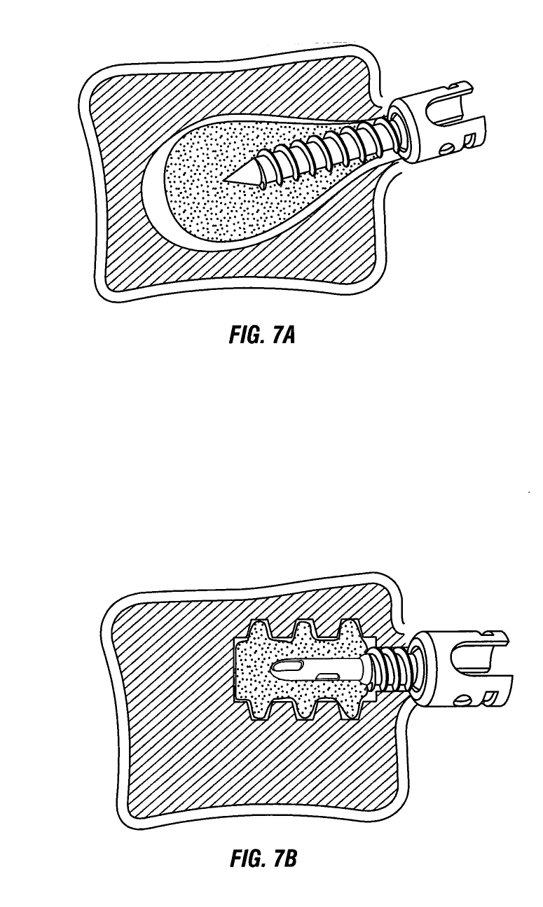

The fenestrated screw includes apertures 114 at the first distal end to allow material (e.g., bone cement) to pass through the shaft of the screw and out of the apertures and into a bone cavity. In certain embodiments, where the fenestrated screw includes an attached containment capsule 101, when the shaft is inserted in a bone cavity and bone cement is passed through the shaft, the containment capsule and cement will conform to the bone cavity wall as illustrated in FIGS. 6a and 6b. In certain embodiments, the containment capsule will interdigitate with cancellous bone.

In certain embodiments, the receiving segment 116 has an inner surface with a guide and advancement structure and a smooth outer surface. In other embodiments, the capture portion includes an open channel 119 adapted to receive a rod implant.

The fenestrated screws of the invention may include polyaxial screws, expandable screws, which when inserted or screwed into the bone and expand by way of some type of expansion mechanism, conventional screws, and the like. In one embodiment, the fenestrated screws can be coated with any number of suitable osteoinductive or osteoconductive materials to enhance fixation in the bone. In another embodiment, the fenestrated screws coating enhance bony in-growth or to further anchor the screw to the bone.

In another embodiment, the fenestrated screws include a containment skin or capsule, such as the one shown in FIG. 1. The fenestrated screws preferably include a linear shaft segment 110 with a tapered tip 112 and a plurality of apertures 114, and a receiving segment 116. The receiving segment 116 may be a separate, attachable unit to be added after the fenestrated screw is inserted into the bone or can be a continuation of the fenestrated screw.

The insertion segment preferably has a tapered tip 112 for easier insertion into the bone. In one embodiment, the tapered tip defines an angle of 60° or less, preferably about 45° or less. The plurality of apertures 114 extend laterally along the linear shaft segment 110. One of ordinary skill in the art would be aware of how to select the size, angle, and length of the fenestrated screw so as to provide optimal bone fastening.

The receiving segment 116 of the fenestrated screw may be fashioned in a number of ways, all of which preferably involve providing the receiving segment 116 the ability to accept the flexible central portion of the system. For example, the receiving segment 116 may be designed to be top loading, side loading, eye-hole loading, and the like. FIG. 1 illustrates a top-loading head 119 that makes up a single body with the shaft of the fenestrated screw.

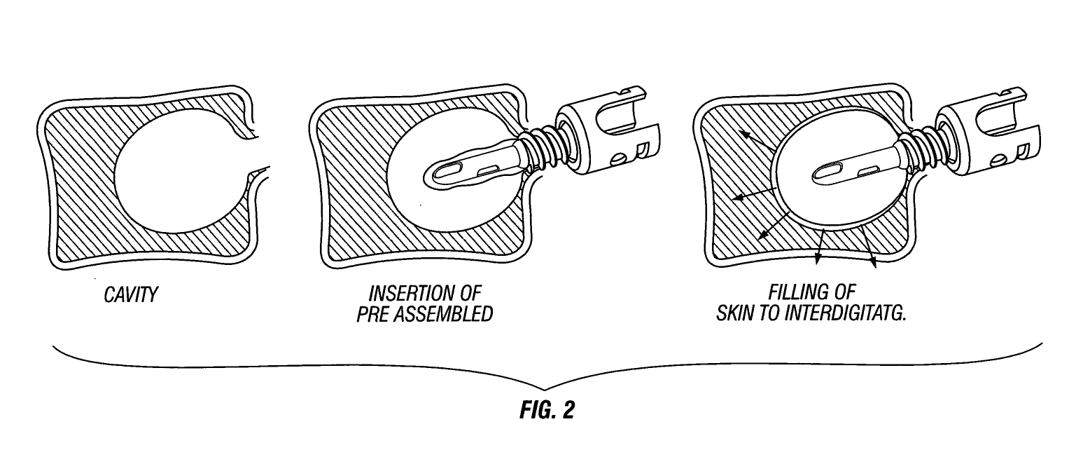

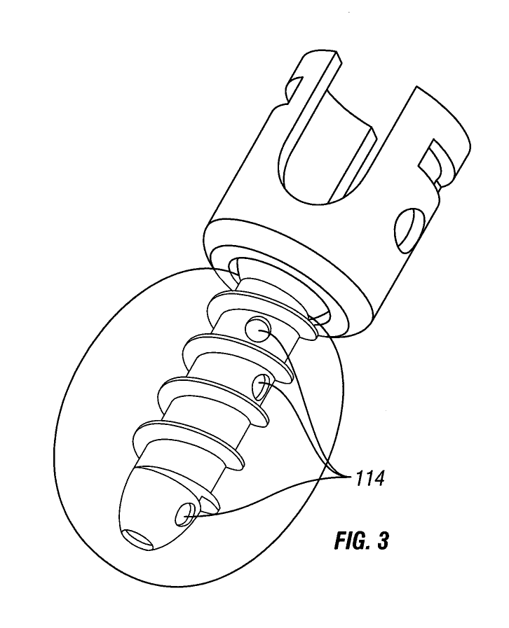

The fenestrated screw shaft may be completely threaded as illustrated in FIG. 2 or partially threaded as illustrated in FIG. 3. Partially threaded fenestrated screws may also include a needle tip as illustrated in FIGS. 1 and 3.

The fenestrated screws may be made from a host of materials. For example, the fenestrated screws may be formed from natural/biological materials, such as allograft, xenograft, and cortical bone. The fenestrated screws may also be formed from synthetic bio-resorbable materials, such as polyanhydride, polyactide, polyglycolide, polyorthoester, polyphosphazene, calcium phosphate, hydroxyapatite, bioactive glass, tyrosine-derived polycarbonate, and mixtures thereof.

In other embodiments, the fenestrated screw of the invention is formed of any suitable biocompatible material(s) of sufficient strength to allow the screw to be implanted into a bone. Exemplary biocompatible materials include (1) metals (for example, titanium or titanium alloys, alloys with cobalt and chromium (cobalt-chrome), stainless steel); (2) plastics (for example, ultra-high molecular weight polyethylene (UHMWPE), polymethylmethacrylate (PMMA), polytetrafluoroethylene (PTFE), polyetheretherketone (PEEK), nylon, polypropylene, and/or PMMA/polyhydroxy-ethylmethacrylate (PHEMA)); (3) ceramics (for example, alumina, beryllia, calcium phosphate, and/or zirconia, among others); (4) composites; (5) bio-resorbable (bioabsorbable) materials or polymers (for example, polymers of alpha-hydroxy carboxylic acids (e.g., polylactic acid (such as PLLA, PDLLA, and/or PDLA), polyglycolic acid, lactide/glycolide copolymers), polydioxanones, polycaprolactones, polytrimethylene carbonate, polyethylene oxide, poly-beta-hydroxybutyrate, poly-.beta.-hydroxypropionate, poly-delta-valerolactone, other bio-resorbable polyesters, and/or natural polymers (such as collagen or other polypeptides, polysaccharides (e.g., starch, cellulose, and/or chitosan), any copolymers thereof); (6) bone tissue (e.g., bone powder and/or bone fragments); and/or the like. In some embodiments, these materials may form the core of the fenestrated screw and/or a coating thereon. The materials may be partially or completely bio-resorbable, as desired or appropriate. In other embodiments, the materials may be partially or completely implantable, as desired or appropriate.

In addition, the fenestrated screws may include growth factors for bone ingrowth and bony attachment, or for soft tissue ingrowth. Non-limiting examples of growth factors include insulin-like growth factor 1, basic fibroblast growth factor, transforming growth factor .beta.-1, platelet-derived growth factor, bone-derived growth factors, arginine, bone morphogenetic protein, LIM mineralization protein, and combinations thereof.

The fenestrated screws of the invention also include a bio-resorbable or implantable containment capsule or skin 101 as illustrated in FIG. 1. In certain embodiments, the containment skin surrounds or encapsulates at least a portion of the fenestrated screw of the invention as illustrated in FIG. 1. In other embodiments, the containment skin can be inserted into a bone cavity and then filled with bone cement as illustrated in FIGS. 6a and 6b.

The bio-resorbable or implantable containment skin can be comprised of any material that is safe when inserted into a bone cavity, for example, a cavity which is created in the spine of a subject.

In other embodiments, the bio-resorbable or implantable containment skin is pressure-resistant.

In other embodiments, the bio-resorbable or implantable containment skin is flexible or stretchable.

In other embodiments, the bio-resorbable or implantable containment skin is comprised of collagen.

In certain embodiments, the containment capsule or skin is a bio-resorbable or implantable, pressure-resistant, flexible collagen capsule.

In various embodiments, the compositions that comprise the containment skin or capsule include any materials that will provide stability to the bone of an osteoporotic patient. In one embodiment, the containment skin reduces the risk of loosening of an instrument anchored in a bone, for example, a pedicle screw. In another embodiment, the containment skin reduces the risk of a windshield-wiping effect, which is common as the bone density of a treated subject decreases (e.g., as in an osteoporotic patient).

Materials that make up the containment skin or capsule include, but are not limited to, bio-resorbable collagen, poly-L-lactide (PLLA), poly-L/D-lactide (PLDLA; a mixture of poly-L-lactic acid with, for example, 30% of poly-D-lactic acid), polyanhydride, polyactide, polyglycolide, polyorthoester, polyphosphazene, calcium phosphate, hydroxyapatite, bioactive glass, tyrosine-derived polycarbonate, and mixtures thereof.

In certain embodiments, the containment skin is bio-resorbable or implantable and has an elasticity modulus similar to that of vertebral bone. In certain embodiments, the bio-resorbable or implantable containment skins reduce the occurrence of stress shielding of the transplant material because, with the resorbing of the material by the body, the newly formed bone material will gradually be loaded more and more.

In another embodiment, the bio-resorbable or implantable containment skin is a porous matrix in which an osteoinductive composition comprising, for example, calcium phosphate and stem cells from adipose tissue is present. In certain embodiments, this porous matrix comprises PLDLA and the calcium phosphate is bicalcium phosphate. The osteoinductive composition may further comprise a growth factor including, but not limited to, insulin-like growth factor 1, basic fibroblast growth factor, transforming growth factor-β-1 (TGF-β), platelet-derived growth factor, bone-derived growth factors, arginine, bone morphogenetic protein (BMPs), LIM mineralization protein (LMP-1), Osf-1, and combinations thereof.

Accordingly, in certain embodiments, the containment skin is coated with bone growth factors, which are specific proteins that influence synthesis and degradation processes in the body by regulating cell growth and cell function. The invention contemplates coating the containment skin with growth factors involved in the formation of bone, for example, the bone-inducing and the bone-producing growth factors. In another embodiment, bone-inducing (i.e., new bone development in a bone-free environment) growth factors, including the bone morphogenetic proteins (BMPs) can make an undifferentiated stem cell differentiate to a progenitor bone cell (i.e., preosteoblast). The bone-producing growth factors of the invention include transforming growth factor-β (TGF-beta), insulin-like growth factor (IGF), and platelet-derived growth factor (PDGF), which make the number of differentiated bone cells increase in number (proliferate) or differentiate further to a bone matrix-forming cell (osteoblast).

In certain embodiments, the containment skin materials are radiolucent, so that follow-up by means of X-rays and/or CT and MRI scans is possible.

The invention encompasses a fenestrated screw including a containment skin for use in implantation of screws in a bone cavity of a subject requiring stabilization or immobilization of one or more bones (e.g., vertebrae). The containment skin of the invention is specifically advantageous for a patient with osteoporosis because it allows improved anchoring to the bone in a patient suffering from decreased bone density.

While the fenestrated screw and containment skin or capsule is preferably a fenestrated pedicle screw, the fenestrated screw of the invention can be implanted and used to fasten various different types of bones.

In certain embodiments, the invention encompasses a generic fenestrated bone screw including a containment capsule or skin useful for fixing or pre-mounting a fastening device on the bone screw, which can be implanted by minimal invasive surgery where appropriate.

In certain embodiments, included on the fenestrated screw is a fastening device, where the fastening device displays an extension that can engage the fenestrated bone screw. In other embodiments, the fenestrated screw and the fastening device comprise a single body.

In certain embodiments, the fenestrated screw head illustrated in FIG. 1 can include a receiving segment 116 differing from a threaded section, with a longitudinal axis, designed for non-sliding accommodation of a retaining area of a fastening device laterally to the longitudinal axis of the recess, where the recess is designed in such a way that it can accommodate a retaining area of a fastening device that engages the recess and is of essentially congruent design.

In certain embodiments, the receiving segment 116 in the fenestrated screw head for accommodating the fastening device is of essentially cylindrical design. In other embodiments, the fastening device is of essentially a conical design.

In another embodiment, the invention encompasses methods for treating an subject requiring bone containment comprising:

a. creating a cavity in a vertebral body;

b. inserting into the cavity a fenestrated screw including a bio-resorbable or implantable containment capsule; and

c. filling the bio-resorbable or implantable containment capsule through the apertures in the fenestrated screw with cement, for example, polymethylmethacrylate, to interdigitate with the cancellous bone, which allows the flexible containment capsule to conform to the cavity.

In certain embodiments, the fenestrated screw can be removed from the bone by an ultrasonic inducer probe, a mechanical removal device, or combinations thereof.

The invention also contemplates kits including various fenestrated screws of the invention as wells as the tools needed for surgery, for example, flexible segment portions or rigid rods for use in spinal surgery. The fenestrated screws may be manufactured in varying widths and lengths to accommodate the type of surgery and needs of the surgeon.

The kits of the invention are intended to broaden a surgeon's options once in surgery to provide a patient with the most optimal stabilization system. By containing the most common fenestrated screws, instrument needs and options for assembling an actual spine stabilization, the revisions are completed faster and with fewer sets from central supply. Overall, there is less of a labor burden on hospital staff and procedure time is reduced by having all required instruments in one convenient kit.

### EXAMPLES

### Example 1

### Bio-resorbable Capsule Contained Cement Pedicle Screw System for Osteoporotic Patients

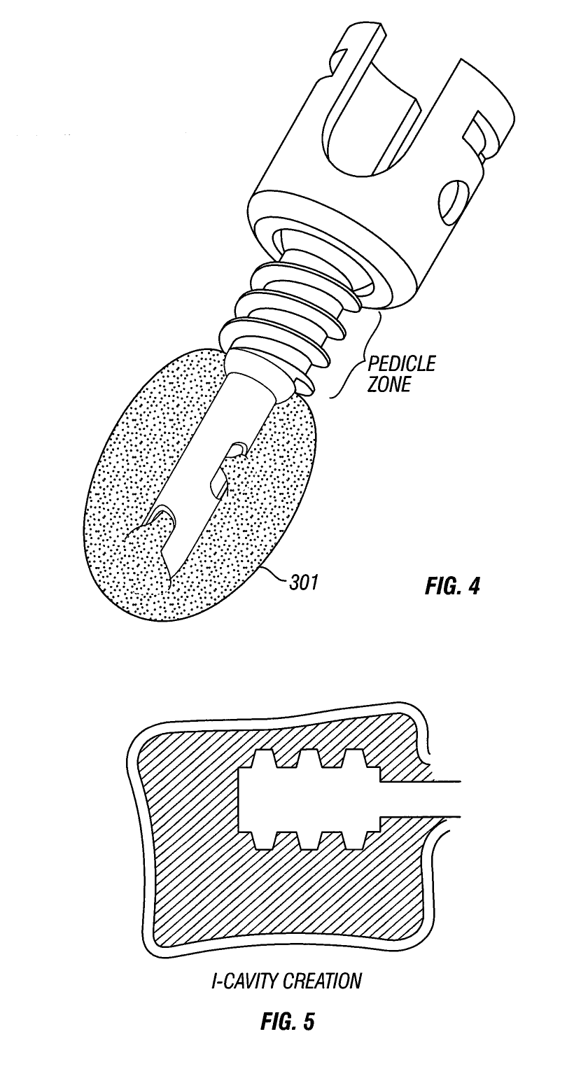

Implantation of the Pedicle Screw into a vertebra cavity including a collagen capsule and bone cement is illustrated in FIGS. 5, 6a and 6b.

A cavity is first created in one or more vertebrae to be stabilized.

A bio-resorbable collagen capsule is then inserted into the vertebra cavity.

Bone cement is then inserted into the collagen capsule, which is in the vertebra cavity allowing the collagen capsule and bone cement to conform to the cavity.

A pedicle screw is then inserted. The insertion parameters are dependent on the patient and readily determinable by the doctor performing the surgery.

This is repeated on multiple vertebrae.

A device, for example, a dynamic spine system is then secured to the pedicle screw.

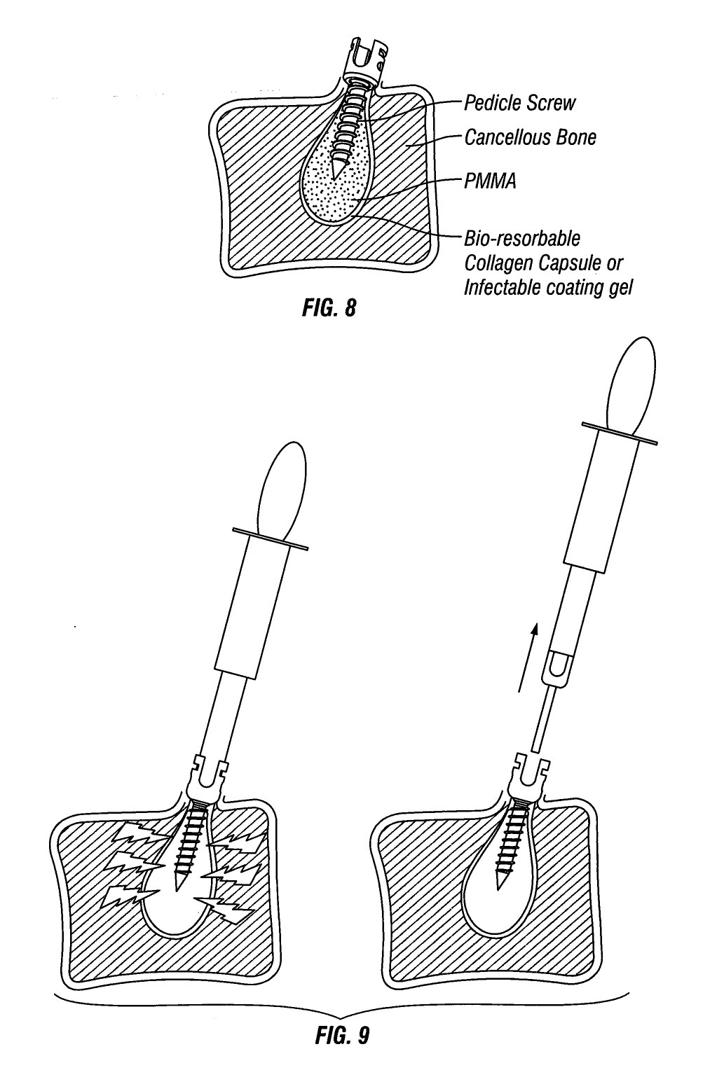

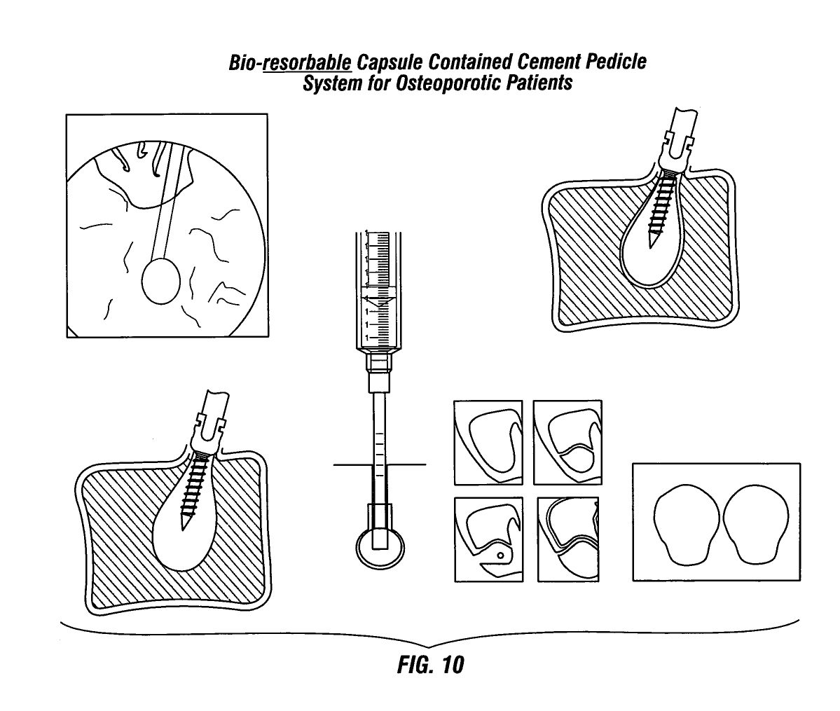

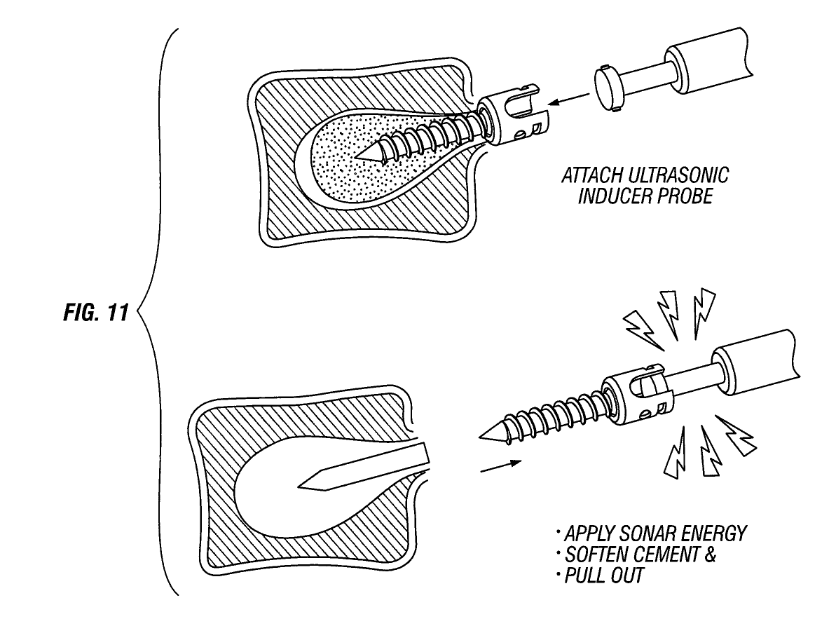

After treatment is complete, the pedicle screw can be removed by attaching an ultrasonic inducer probe and applying sonar energy to soften the cement and pulling out the pedicle screw as illustrated in FIGS. 8 and 10. The pedicle screw can also be removed by mechanical removal such as removal of the implant and cement bolus using a bone trocar to grind the cement away and remove the implant in a cylindrical like core piece.

### Example 2

### Pedicle Screw Containing Bio-Resorbable Capsule

A cavity is first created in one or more vertebrae to be stabilized.

A bio-resorbable collagen capsule, which is attached to a fenestrated screw, is inserted into the vertebra cavity.

Bone cement is then passed through the fenestrated screw and into the collagen capsule, which is in the vertebral cavity allowing the collagen capsule and bone cement to conform to the cavity.

The insertion parameters are dependent on the patient and readily determinable by the doctor performing the surgery.

This may be repeated on multiple vertebrae.

A device, for example, a dynamic spine system is then secured to the pedicle screw.

After treatment is complete, the pedicle screw can be removed by attaching an ultrasonic inducer probe and applying sonar energy to soften the cement and pulling out the pedicle screw as illustrated in FIGS. 8 and 10. The pedicle screw can also be removed by mechanical removal such as removal of the implant and cement bolus using a bone trocar to grind the cement away and remove the implant in a cylindrical like core piece.

In the specification, there have been disclosed typical preferred embodiments of the invention and, although specific terms are employed, they are used in a generic and descriptive sense only and not for purposes of limitation. Obviously many modifications and variations of the invention are possible in light of the above teachings. It is therefore to be understood that the invention may be practiced otherwise than as specifically described.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited above are incorporated herein by reference to the extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A fenestrated screw comprising:
a bio-resorbable or implantable containment skin;
at least one aperture located on a first distal end of the fenestrated screw; and
a second distal end configured with a receiving segment to allow attachment to a stabilizing system or device;

2. The fenestrated screw of claim 1, wherein the containment skin encapsulates one or more apertures on the fenestrated screw.

3. The fenestrated screw of claim 1, wherein the bio-resorbable containment skin comprises collagen, poly-L-lactide, poly-L/D-lactide, polyanhydride, polyactide, polyglycolide, polyorthoester, polyphosphazene, calcium phosphate, hydroxyapatite, bioactive glass, tyrosine-derived polycarbonate, silicon, thermoplastic polycarbonate urethane, and mixtures thereof and mixtures thereof.

4. The fenestrated screw of claim 1, wherein the implantable containment skin comprises collagen, poly-L-lactide, poly-L/D-lactide, polyanhydride, polyactide, polyglycolide, polyorthoester, polyphosphazene, calcium phosphate, hydroxyapatite, bioactive glass, tyrosine-derived polycarbonate, silicon, thermoplastic polycarbonate urethane, and mixtures thereof and mixtures thereof.

5. The fenestrated screw of claim 1, wherein the bio-resorbable containment skin is comprised of a bio-resorbable collagen capsule or injectable coating gel material.

6. The fenestrated screw of claim 1, wherein the containment skin is comprised of a pressure-resistant material.

7. The fenestrated screw of claim 1, wherein the containment skin is comprised of a polymer material.

8. The fenestrated screw of claim 1, wherein the stabilizing system or device comprises one or more of screws and cords; screws, cords, and spacers; screws and flexible rods; screws and rods with movable parts; flexible screws and flexible rods; screws and inflatable rods; and combinations thereof.

9. The fenestrated screw of claim 1, wherein the containment skin is coated with a bone growth agent.

10. The fenestrated screw of claim 1, wherein the bone growth agent comprises insulin-like growth factor 1, basic fibroblast growth factor, transforming growth factor-β-1 (TGF-β, platelet-derived growth factor, bone-derived growth factors, arginine, bone morphogenetic protein (BMPs), LIM mineralization protein (LMP-1), Osf-1, and combinations thereof.

11. A method of treating an subject requiring bone containment comprising:
creating a cavity in a bone;
inserting one or more bio-resorbable capsule(s);
inserting bone cement into the cavity; and
inserting one or more screw(s) into the cavity.

12. The method of claim 11, wherein the bio-resorbable capsule when filled with cement conforms to the cavity wall.

13. The method of claim 11, wherein the inserting one or more bio-resorbable capsule(s) and inserting bone cement into the cavity are achieved using a fenestrated screw comprising one or more aperture, wherein the bio-resorbable capsule(s) encapsulates one or more apertures on the fenestrated screw.

14. The method of claim 11, wherein the fenestrated screw is a pedicle screw.

15. The method of claim 11, further comprising:
attaching to the fenestrated screw a stabilizing system or device comprising one or more of screws and cords; screws, cords, and spacers; screws and flexible rods; screws and rods with movable parts; flexible screws and flexible rods; screws and inflatable rods; and combinations thereof.

16. A method for locking relative positions of a first and second vertebrae in a subject suffering from osteoporosis comprising:
providing a first fenestrated screw comprising one or more bio-resorbable capsule(s) attached to the first vertebra;
providing a second fenestrated screw comprising one or more bio-resorbable capsule(s) attached to the second vertebra;
engaging with the first fenestrated screw such that the second fenestrated screw is translatable toward and away from the first member to permit stabilized motion of the second vertebra with respect to the first vertebra, and
a first substantially elongate resilient member positioned within a cavity defined by engagement of the first and second fenestrated screw along a longitudinal axis of the cavity, such that the first and second fenestrated screws cooperate to keep the first resilient fenestrated screw substantially straight; and inserting a first locking component through a plurality of apertures formed in the first fenestrated screw and into contact with a portion of the second fenestrated screw disposed within the first member to lock the first stabilizer to substantially prevent translation of the second fenestrated screw toward and away from the first member, wherein the inserting step includes placing prongs of a clip in the apertures.
